(19) United States
(12) Patent Application Publication
(10) Pub. No.: US 2015/0119893 A1
(43) Pub. Date: Apr. 30, 2015

(54) Method And Apparatus For Preparing An Implantation Site

(71) Applicant: Biomet Manufacturing, LLC, Warsaw, IN (US)

(72) Inventor: Tyler D. WITT, Warsaw, IN (US)

(73) Assignee: Biomet Manufacturing, LLC, Warsaw, IN (US)

(21) Appl. No.: 14/065,718

(22) Filed: Oct. 29, 2013

(51) Int. Cl. A61B 17/16

(52) U.S. Cl. CPC A61B 17/164; A61B 17/1659
USPC 606/85

(57) ABSTRACT

A bone preparation system, such as a broach, includes at least a first member and a second member. The first member and the second member may be connected. Both the first member and the second member, when attached to the first member, are configured to directly contact and broach an anatomy.

### FIELD

The subject disclosure relates to surgical instrumentation, and particularly to a broach for preparing a long bone for an implantation procedure and prosthesis.

### BACKGROUND

This section provides background information related to the present disclosure which is not necessarily prior art.

A bone in an anatomy may be prepared for a procedure, such as implantation of a prosthesis. Bones can include, long bones such as a femur, tibia, humerus, and other long bones of a human anatomy and equivalent long bones of other anatomies. In preparing long bones for implantation of the prosthesis portions of the bones can be removed, such as an intramedullary canal. For example, preparing the long bone for an implantation generally includes preparing a recess or opening to receive the implant member.

In preparing the long bone for an implant member, a reamer, broach, rasp, or other instrument may be used to remove portions of the interior of the long bone. Generally, at least a portion of the intramedullary canal is removed using a selected instrument. When performing the long bone preparation, a selected length of removal of material is achieved using a plurality of lengths of rasp or broaches. A user selects the appropriate length rasp or broach to be used to remove the material from the material from the interior of the bone.

### SUMMARY

This section provides a general summary of the disclosure, and is not a comprehensive disclosure of its full scope or all of its features.

A bone preparation system, such as a broach, includes at least a first member and a second member. The first member and the second member may be connected. Both the first member and the second member, when attached to the first member, are configured to directly contact and broach an anatomy.

A modular system allows for user selection of a length of removal of an intramedullary canal. The system can include a first or main member that is able to rasp and/or broach a first length of a bone. The system further includes at least one or only one modular members that can be connected to the main member to allow for a second length rasp and/or broach or bone removal. The main member can be used without any additional modular pieces, particularly at a distal end, to directly broach and/or rasp the bone. Accordingly, the main member can be used to perform a broach and/or rasp without any additional distal attachments. It is understood that the main member can be connected to a handle to assist in guiding or moving the main member into the long bone for broaching the bone.

Further, the system can be used to prepare an interior of the bone. The preparation of the interior of the bone can be referred to by any appropriate term. It is understood, herein, that reference to either one of broach, ream, resect, rasp is not intended, unless specified otherwise, to disregard or not allow other types of bone preparation and removal. Thus, the system may be a rasp, a broach, etc.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1A is an exploded view of a broach system including a main body and an extension member;

FIG. 1B is a detailed view of a distal end of the main body of the broach system;

FIG. 2 is an assembled view of the broach system;

FIG. 3 is an environmental view of a main body of the broach system positioned in a long bone;

FIG. 4 is an environmental view of the broach system including the main body and attachment member in a long bone;

FIG. 4A is an environmental view of the broach system including the main body and an attachment member, according to various embodiments, in a long bone; and

FIG. 5 is a kit view of a broach system including a main body and a plurality of extension members.

Corresponding reference numerals indicate corresponding parts throughout the several views of the drawings.

### DETAILED DESCRIPTION

Example embodiments will now be described more fully with reference to the accompanying drawings.

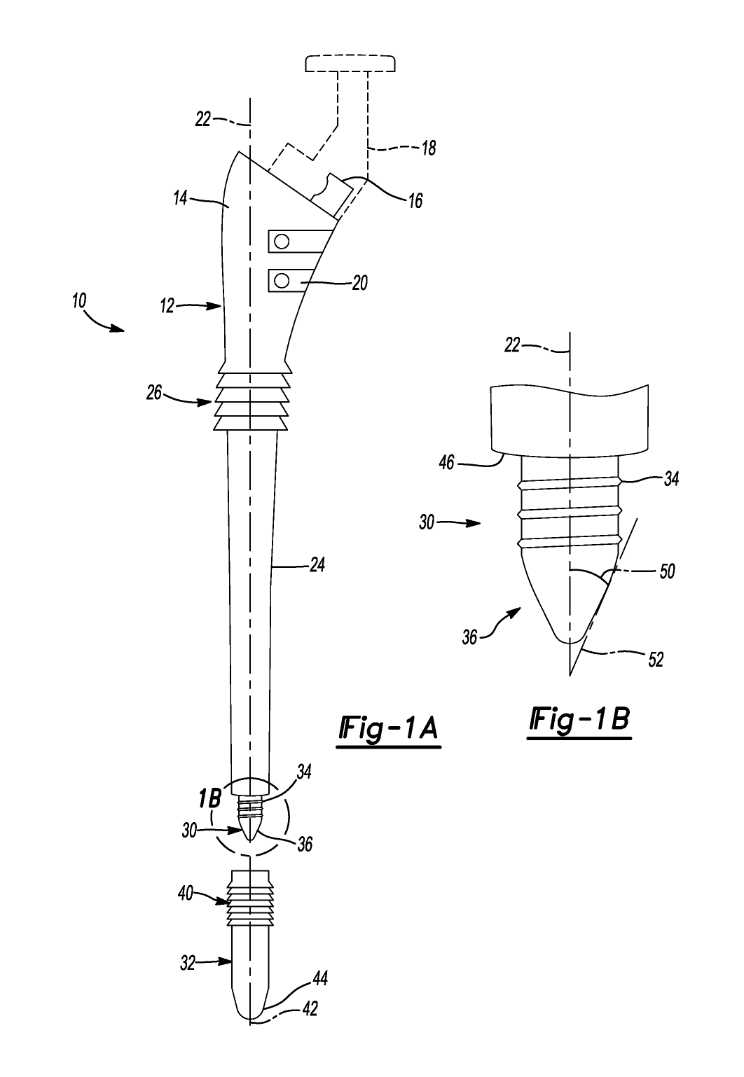

A bone preparation system can include a broach assembly 10 illustrated in FIG. 1A. The broach assembly 10 can include various portions for preparing a bone for implantation of a prosthesis, such as broaching a long bone. In broaching a long bone, portions of an interior of the long bone, such as intramedullary canal, are removed. It is understood, however, that a similar instrument can include a rasp, a file, or other instrumentation that can be driven substantially axially into a long bone for removing or loosening portions of the interior of the long bone. Broaches generally can include selected single broaches such as the Taperloc® Hip System or Vision® Hip System sold by Biomet, Inc. having a place of business in Indiana, USA and the broach used with the Echo® Bi-Metric® Hip Stem or BIO-Moore II Modular Prosthesis System, also sold by Biomet, Inc.

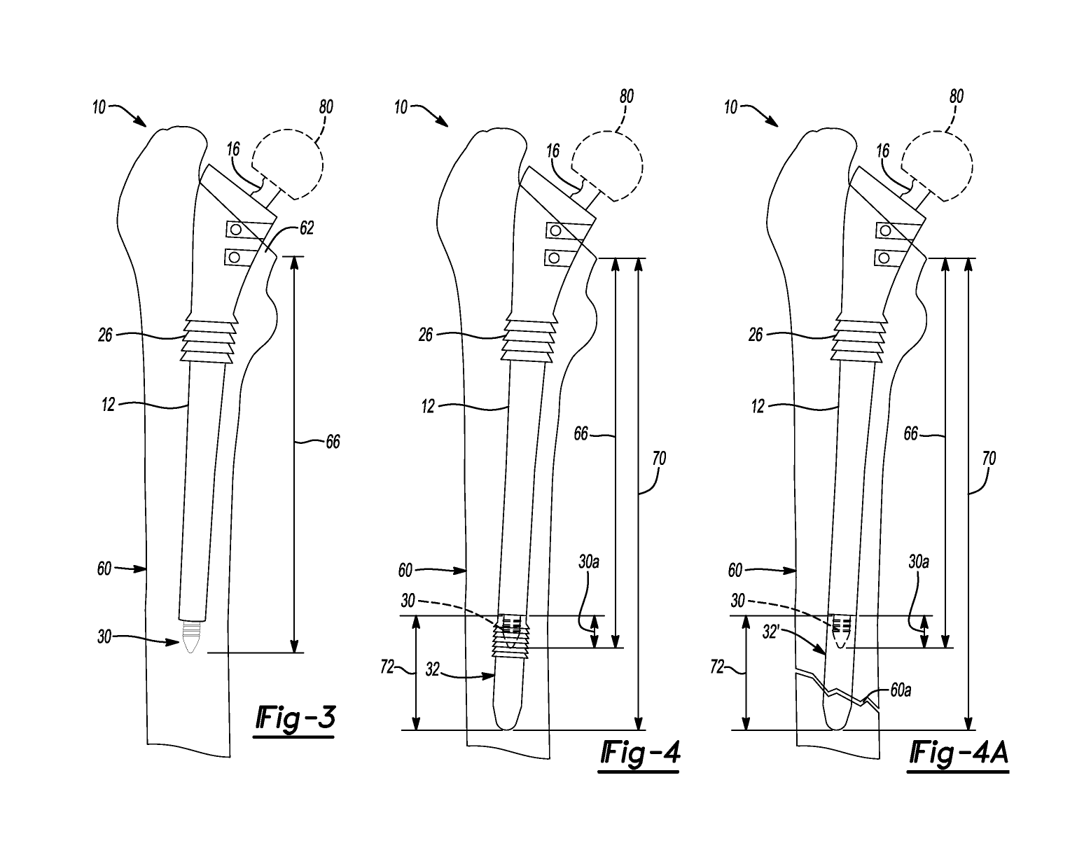

Returning reference to FIG. 1A a first member can include a main body 12. The main body 12 can include an upper or proximal end or portion 14. A handle or neck connection portion 16 can extend or be formed with the proximal portion 14. The connection portion 16 can connect to a handle 18 (shown in phantom). The handle 18 can be driven with a hammer or other appropriate instrument (not specifically illustrated) to drive the broach 10 into the selected anatomy, as discussed further herein. The connection portion 16 can also allow for connection of trial portions, such as a trial neck, a trial head (FIGS. 3 and 4), or other trial members to be trialed when positioned within the anatomy. Trialing using the broach system 10 can allow for determination of an appropriate head size, neck length, or other selected features of a final prosthetic system. The final or implanted prosthetic system can include a proximal femur implant, a distal femur implant, a humerus implant, or other appropriate prosthesis. The main body 12 can further include one or more slots 20 that can be interconnected with tables or platforms that can contact the anatomy to limit the movement of the broach system 10 into the anatomy.

The main body 12 can extend along a longitudinal axis 22 that extends through the proximal portion 14 and along a shaft portion 24. Formed along a selected portion of the body 12, including an entire length or substantially over at least to the length that the shaft 24, can be a cutting surface. The cutting surface can be one or a plurality of cutting edges or cutting ledges 26. The cutting ledges 26 can scrape along or engage the bone on the interior of the long bone to remove portions of the interior of the long bone. Although illustrated in FIG. 1A is only a short series of the cutting edges 26, it is understood that the cutting edges can extend along substantially the entire length of the main broach body 12, or at least the shaft 24 thereof. It is also understood that the cutting edges 26 can be sharpened and extend any selected length from the central longitudinal axis 22 depending upon the amount of bone or aggressiveness of broaching selected or required within the bone.

Near a distal end of the main body 12 is a connection region or portion 30, discussed in further detail herein, but operable to allow for a connection of a modular extension member 32. The connection portion 30 can be formed as one piece with the main body 12, such as by machining from bar stock, forging, or casting. Alternatively, the connection portion 30 can be connected to the main body 12, such as by welding, brazing or selected adhesive.

The connection portion 30 can include a connecting section, such as a threaded region 34. The threaded region 34 can have external threads that can engage internal threads in the extension member 32. It is understood, however, that the connecting region of the connection portion can include alternative and/or additional connection features such as taper fits, snap fits, etc. The connection portion 30 can also include a distal tip 36. The distal tip 36 can be formed and/or configured to directly engage the bone, as discussed further herein.

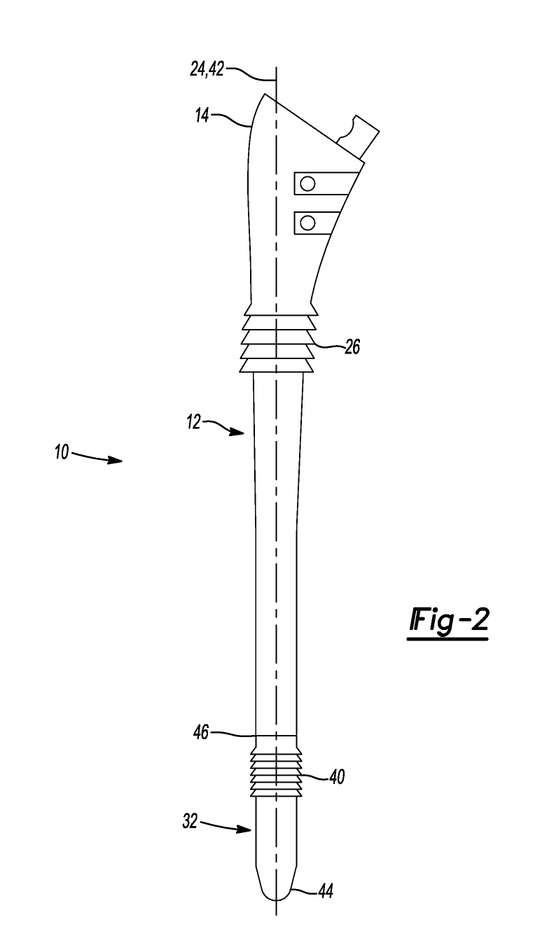

The extension member 32 can then be threaded onto the connection portion 30 to engage the main body 12, as illustrated in FIG. 2. The extension member 32 can also include one or more cutting edges 40. The cutting edges 40 can include a feature similar to the cutting edges 26, discussed above, including a length extending from a central longitudinal axis 42 of the extension member 32 or other selected feature to engage the anatomy for removing selected bone. The extension member 32 can also include a distal end 44 that can taper towards the central longitudinal axis 42. When connected, the cutting edges 40 of the extension can form a single continuous cutting surface with the cutting edges 26 of the main body 12.

Illustrated in FIG. 1B is the connection region 30. As discussed above, the connection portion 30 can include threads 34 in the connection region to engage internal threads of the extension member 32. A ledge or shoulder 46 defines an extent of the connection region, and generally is at a proximal end of the connection region 30. The ledge 46 can engage the extension member 32 to limit travel of the extension member 32.

The tapered tip 36 can also have a curved outer surface or a substantially flat or straight outer surface. The outer surface of the tip 36, however, can define a taper angle 50 between the central axis 22 and a line 52 that is either tangent to the exterior of the tapered tip 36 or is defined as a line extending along the flat tapered surface 36. As discussed further herein, this allows the tapered distal end 36 of the connection region 30 to be positioned within a portion of the long bone in an appropriate manner to allow for broaching of the long bone with the main body 12 alone without connection of any other members, including the extension member 32.

With continuing reference to FIGS. 1A and 1B, and further reference to FIG. 2, the assembly 10 can be assembled, as specifically illustrated in FIG. 2, for use as a combined system. As illustrated in FIG. 2 the extension member 32 can be connected to the main body 12 to form a substantially continuous broach assembly extending from the proximal portion 14 to the distal tip 44 of the extension member 32. The assembly 10 can extend along the assembled longitudinal axis 22, 42 such that the assembly 10, in the assembled state, will broach a single area that is substantially continuous along a combined length of the body 12 and the extension member 32. Further, the transition between the main body 12 and the extension member 32 is substantially congruent and smooth. Accordingly, the exterior of the extension member 32 at the boundary with the main body 12, allows the extension member 32 to engage the shoulder 46 of the main body 12 such that the main body 12 and the extension member 32 form a single unit. It is understood, however, that the connection or the boundary between the extension member 32 and the main body 12 can also be non-congruent such that it can form a cutting edge or different size rasp region. Regardless, the assembly can be formed to broach a bone along the combined and generally aligned axes 22, 42.

Further, by the connection of the extension member 32 with the main body 12, the cutting edges 40 of the extension member 32 can form a cutting surface or exterior cutting boundary with the cutting edge 26 of the main body 12. Again, it is understood, that the outer or external cutting surface can be substantially parallel, cylindrical, or may be tapered such that the cutting diameter at the distal end 42 of the extension member 32 is less than the cutting diameter near the proximal end 14 of the main body 12. Further, it is understood that the extension member 32 can be connected to the main body 12 in any appropriate manner including by the interaction with the threads 34, as discussed above or with other appropriate connection mechanisms. For example, a snap connection, a taper fit connection or a snapfit connection.

With reference to FIGS. 3 and 4 the broach assembly 10 is illustrated during use. With specific reference to FIG. 3, the main body 12 can be used alone to broach a long bone, such as a femur 60. The femur 60 can be prepared in an appropriate manner, such as resection of a femoral head and neck to provide a resected surface 62. The main body 12 can then be inserted into the femur 60, such as into an IM portion of the femur 60, using appropriate mechanisms, as discussed above. For example, the handle 18 can be connected to the connection portion 16 and the main body 12 can be driven into the femur 60. The main body 12 alone can broach a portion of the femur 60 due to the interaction of the cutting edges 26 with the interior of the femur 60 and the shape and configuration of the attachment portion 30 at the distal end of the main body 12. As discussed above, the terminal end of the attachment portion 30 can include a taper, curve, or other appropriate configuration to allow the main body 12 alone to be inserted and broach the femur 60.

Alone, the main body 12 can be used to broach a selected length or distance 66 into the bone of the femur 60, as illustrated in FIG. 3. The distance 66 can be any appropriate length, such as a main body length 66 that is a length of the main body 12. The main body length 66 can be less than a combined length or distance 70, as illustrated in FIG. 4. Accordingly, based upon a pre-planning, surgeon selection, or other requirement for a procedure, the main body 12 can be used to broach the femur 60 to the first length 66. The first length 66 may be selected based upon the size of the implant to be implanted into the femur 60, the size of the patient being broached, or other appropriate considerations. Additionally, the main body 12 can receive a trial head 80 (shown in phantom) for trialing after placement of the main body 12 within the femur 60. It is understood that other trial portions can also be connected with the connection portion 16 according to various trialing techniques.

With specific reference to FIG. 4, the second or attached distance 70 can include a distance that is greater than the first distance 66 and can be defined as a broach distance or length based upon the length of the main body 12 and the extension member 32, which can be connected to the main body 12, as illustrated in FIG. 4. The second distance 70 can include a combination of the first distance 66 and an attachment or extension distance 72 minus a distance 30a of the attachment portion 30. The attachment distance 72 can be dependent upon the length of the extension member 32 and can also be selected for various procedures, such as a length of the patient's femur 60, an implant to be positioned within the femur 60, or other considerations. Nevertheless, the connection of the extension member 32 to the main body 12 can be used to form the assembly 10 that can broach the second length 70 into the femur 60. Additionally, a trial head 80 or other trial portion can be interconnected with the connection portion 16.

With additional reference to FIG. 4A, according to various embodiments, a smooth extension 32′ can be connected with the main body 12. The extension 32′ can be made with the attachment portion 30 in any appropriate manner, including those discussed above. The smooth extension 32′ can have an exterior surface that is substantially smooth and does not include any of the cutting edges 40 as illustrated and included in the extension 32. Accordingly, the smooth extension 32′ may be included to not broach or cut any bone, such as the femur 60, but can be positioned within the bone 60 to assist in stabilization of the main body 12 of the broach assembly. Accordingly, the smooth extension 32′ can be connected with the main body 12 to provide for a selected length of a combination of main body 12 and the extension 32′ without broaching a distal portion or extension into the femur 60. According to various embodiments, as is generally understood by one skilled in the art, a reamer or other instrument can ream a portion of the bone 60. For example, the bone 60 may have a fracture or weakened portion 60a and the extension 32′ can extend beyond the weakened or fractured portion 60a, as illustrated in FIG. 4A. Accordingly, the smooth extension 32′ can engage a portion of the bone 60, such as a portion passed the fracture 60a, for stabilizing the assembled broach assembly during a procedure. It may be selected by a user to provide the smooth extension 32′ to engage a portion of the bone 60 passed the fracture portion 60a for stabilizing the assembled broach assembly and/or selecting a final length of an implant, such as a femoral stem.

As discussed above, the main body 12 can be used alone to broach an appropriate bone, such as the femur 60. The connection portion 30 of the main body 12 can interact with the femur 60 alone without any member placed thereon. Accordingly, the main body 12 can be used alone to broach a selected distance, such as the first distance 66 into the bone 60. Only a single member, such as the extension member 32, need be interconnected with the main body 12 to broach the second distance 70. It is understood that the assembly 10 may include other connections, such as an intermediate connection between the extension member 32 and the main body 12 or have a plurality of extension members 32 of the varying lengths.

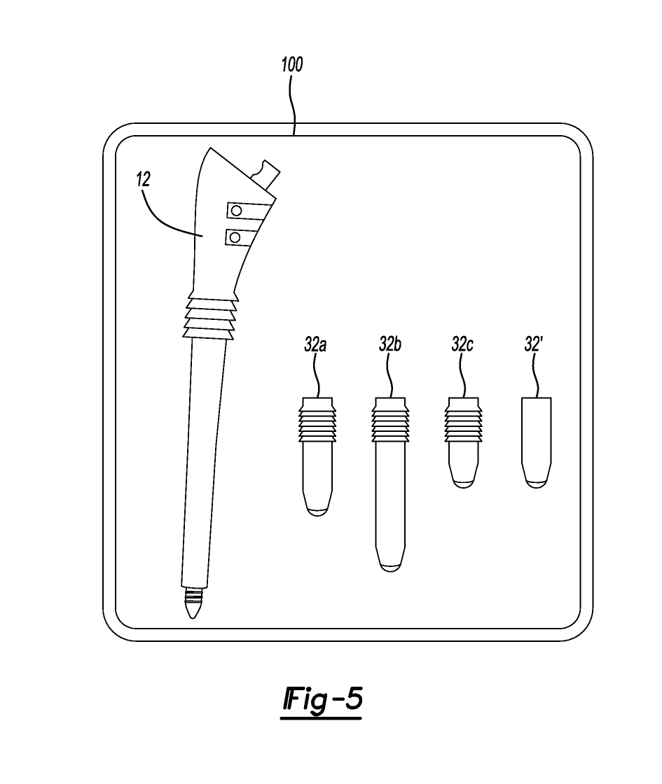

For example, as illustrated in FIG. 5, a kit or system 100 can include the main body 12 and a plurality of the extension member 32, including extension members 32a, 32b, and 32c. Each of the extension members 32 can include different lengths such that the attachment length 72 may be varied depending upon the selected extension members 32 connected to the main body 12. For example, the extension member 32a can have a first distance that is less than a second length of the second extension member 32b while the first distance of the first extension member 32a can be greater than a third length of the third extension member 32c. Thus, based upon the selection of the extension members 32a, 32b, and 32c, the second length 70 can be varied. Also, according to various embodiments, one or more of the smooth extensions 32′ can be included within the kit 100. Thus, the single kit 100 can include broach extensions for cutting and/or for stabilizing and not cutting the bone 60. A user can select the length for broaching and/or trialing. It is also understood that the extension members 32 can be connected to the main body 12 after the beginning of a procedure based upon a user selection, such as confirming a length broached within the femur 60

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An assembly to broach an anatomy, comprising:
a first member extending from a first end to a second end;
a first member cutting portion formed along at least a portion of a length of the first member between the first end and the second end;
a connection portion extending from the second end, the connection portion extending from the second end to a connection member terminal end;
wherein the connection member terminal end is tapered relative to a connection member proximal end adjacent to the second end of the first member;
wherein the first member cutting portion is configured to prepare the anatomy for an implant.

2. The assembly of claim 1, further comprising:
a broach extension member having an extension member cutting portion;
wherein the broach extension member is configured to connect to the connection portion of the first member.

3. The assembly of claim 2, wherein the connection member includes an external thread and the broach extension member has an internal thread, wherein the external thread and the internal thread interact to connect the broach extension member to the first member.

4. The assembly of claim 2, wherein the first member cutting portion and the extension member cutting portion cooperate to form a single cutting configuration of the connected first member and the broach extension member.

5. The assembly of claim 4, wherein the first member is tapered from the first end to the second end.

6. The assembly of claim 4, wherein a first member external perimeter of the first member at the second end is substantially equivalent to a broach extension member external perimeter of the broach extension member.

7. The assembly of claim 1, wherein the connection portion is configured to be driven directly into a bone of the anatomy.

8. The assembly of claim 1, further comprising:
a broach extension member having a smooth external surface;
wherein the broach extension member is configured to connect to the connection portion of the first member.

9. An assembly to broach an anatomy, comprising:
a first member extending from a first end to a second end, the first member having,
a first member cutting portion formed along at least a portion of a length of the first member between the first end and the second end;
a connection portion extending from the second end, the connection portion extending from the second end to a connection member terminal end and configured to be driven directly into a bone of the anatomy;
wherein the connection member terminal end is tapered relative to a connection member proximal end adjacent to the second end of the first member;
wherein the first member cutting portion is configured to prepare the anatomy for an implant; and
a broach extension member selectively attachable to the connection portion of the first member, wherein the broach extension is configured to be driven directly into the bone of the anatomy when connected to the first member.

10. The assembly of claim 9, wherein the connection portion has a first connection and the broach extension has a second connection such that the first connection and the second connection connect to selectively fix the broach extension to the first member.

11. The assembly of claim 10, wherein the first connection is an external thread and the second connection is an internal thread, wherein the external thread and the internal thread interact to connect the broach extension member to the first member.

12. The assembly of claim 10, wherein the broach extension member includes an extension cutting portion.

13. The assembly of claim 12, wherein the first member cutting portion and the extension member cutting portion cooperate to form a single cutting configuration of the connected first member and the broach extension member.

14. The assembly of claim 10, wherein the first member and the connection portion are formed as one piece.

15. The assembly of claim 10, wherein the broach extension member includes an external smooth and non-cutting surface.

16. A method of providing a broach for preparing an anatomy for an implant, the method comprising:
providing a first member extending from a first end to a second end having a first member cutting portion formed along at least a portion of a length of the first member between the first end and the second end;
providing the first member with a connection portion extending from the second end, the connection portion extending from the second end to a connection member terminal end;
configuring the connection member terminal end to be driven directly into a bone of the anatomy;
configuring the connection portion to include a connecting section; and
providing a broach extension member to be selectively attached to the connection portion of the first member with the connecting section.

17. The method of claim 16, further comprising:
configuring the broach extension to be driven directly into the bone of the anatomy when connected to the first member.

18. The method of claim 16, further comprising:
configuring the connection portion terminal end to taper outward towards a connection portion proximal end that is adjacent to the second end of the first member.

19. The method of claim 16, further comprising:
providing a second cutting portion formed along at least a portion of a length of the provided broach extension.

20. The method of claim 19, further comprising:
configuring the first member cutting portion and the second cutting portion to form a substantially continuous cutting region when the provided broach extension is connected to the first member.

21. The method of claim 16, further comprising:
forming the provided first member as one piece with the provided connection portion.

22. The method of claim 16, further comprising:
providing an external smooth and non-cutting surface along at least a portion of a length of the provided broach extension.
